(19) United States
(12) Patent Application Publication
(10) Pub. No.: US 2023/0071396 A1
(43) Pub. Date: Mar. 9, 2023

(54) METHOD OF DETECTING A RESPIRATORY VIRAL INFECTION

(71) Applicant: Breathonix Pte. Ltd., Singapore (SG)

(72) Inventors: Zhunan JIA, Singapore (SG); Fang DU, Singapore (SG); Wei Qiang ONG, Singapore (SG)

(21) Appl. No.: 17/896,801

(22) Filed: Aug. 26, 2022

(30) Foreign Application Priority Data
Aug. 30, 2021 (SG) 10202109466S

(51) Int. Cl. G01N 33/569; G01N 33/497

(52) U.S. Cl. CPC G01N 33/56983; G01N 33/497

(57) ABSTRACT

The present disclosure relates to the field of virology. In particular, the disclosure teaches methods of detecting the presence of a respiratory viral infection (e.g. by a coronavirus or influenza virus) in a subject, and methods of treating said respiratory viral infection.

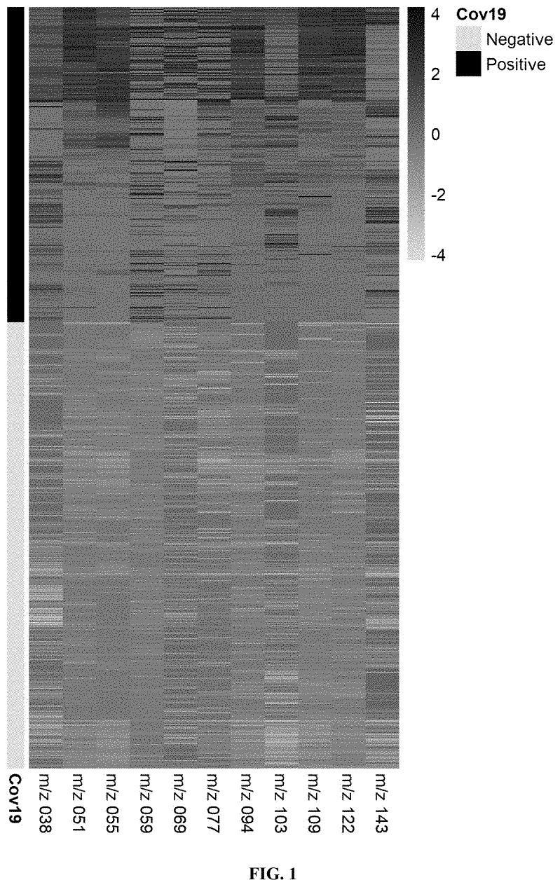

### CROSS-REFERENCE TO RELATED APPLICATIONS

This application claims priority to Singapore Patent Application No. SG 10202109466S filed Aug. 30, 2021, which is incorporated by reference herein in its entirety.

### FIELD

The present disclosure relates to the field of virology. In particular, the disclosure teaches methods of detecting the presence or determining the likelihood of a respiratory viral infection (e.g. by a coronavirus or influenza virus) in a subject, and methods of treating said respiratory viral infection.

### BACKGROUND

Respiratory viral infections are viral infections that affect the respiratory tract. Common viruses that cases respiratory viral infections include coronavirus, influenza virus, respiratory syncytial virus (RSV) and rhinovirus. Many of these viruses are seasonal in prevalence and can be affected by environmental conditions such as humidity and temperature. From time to time, there are novel strains of viruses that lead to a widespread pandemic.

COVID-19 is an respiratory viral infection caused by the SARS-CoV-2 virus that has led to an ongoing global pandemic since early 2020. The pandemic has resulted in about 86 million cases and 1.8 million deaths to date (as of 5 Jan. 2021). The SARS-CoV-2 virus is highly transmissible and can be contacted through close contact with an infected person through the respiratory route. Common symptoms of COVID-19 include fever, cough, shortness of breath and fatigue, although the type and severity of symptoms can vary dramatically between patients. Furthermore, a number of infected patients remain asymptomatic. There is a lack of effective treatments for COVID-19, although there has been a number of therapeutic drugs that have been recently approved. Current measures to contain the spread of COVID-19 include wearing of face masks, social distancing, vaccination as well as monitoring and isolating infected patients.

There is a need for effective methods to rapidly determine whether a subject is suffering from respiratory viral infection, such as during a pandemic. A common method that is currently used is a real-time reverse transcription polymerase chain reaction (rRT-PCR) method for detecting SARS-CoV-2 virus in a subject. However, this method is relatively expensive and time consuming, taking up to a few hours to obtain results. This method also requires respiratory samples such as nasopharyngeal or nasal swabs to be taken from a subject, which can be uncomfortable and painful.

Accordingly, there is a need to overcome, or at least to alleviate, one or more of the above mentioned problems.

### SUMMARY

Disclosed herein is a method of detecting the presence or determining the likelihood of a respiratory viral infection in a subject, the method comprising identifying one or more spectral peaks in a mass spectrum of a breath sample from the subject, wherein the presence or likelihood of the respiratory viral infection is characterized by identifying at least one spectral peak at about 38 m/z, about 51 m/z, about 55 m/z, about 59 m/z, about 69 m/z, about 77 m/z, about 94 m/z, about 103 m/z, about 109 m/z, about 122 m/z and about 143 m/z, wherein the intensity of the one or more spectral peaks, or a value derived therefrom, as compared to a reference detects the presence or predicts the likelihood of the respiratory viral infection in the subject.

Disclosed herein is a method of detecting the presence or determining the likelihood of a respiratory viral infection in a subject, the method comprising conducting a mass spectral analysis on a sample from the subject, wherein the presence or likelihood of a respiratory viral infection is characterized by identifying at least one spectral peak at about 38 m/z, about 51 m/z, about 55 m/z, about 59 m/z, about 69 m/z, about 77 m/z, about 94 m/z, about 103 m/z, about 109 m/z, about 122 m/z and about 143 m/z, wherein the intensity of the one or more spectral peaks, or a value derived therefrom, as compared to a reference detects the presence or predicts the likelihood of the respiratory viral infection in the subject.

Disclosed herein is a method of treating a respiratory viral infection in a subject, the method comprising a) identifying one or more spectral peaks in a mass spectrum of a breath sample from the subject, wherein the presence or likelihood of the respiratory viral infection is characterized by identifying at least one spectral peak at about 38 m/z, about 51 m/z, about 55 m/z, about 59 m/z, about 69 m/z, about 77 m/z, about 94 m/z, about 103 m/z, about 109 m/z, about 122 m/z and about 143 m/z, wherein the intensity of the one or more spectral peaks, or a value derived therefrom, as compared to a reference detects the presence or predicts the likelihood of the respiratory viral infection in the subject, and b) administering a therapeutically effective amount of a anti-viral agent to the subject.

Disclosed herein is a method of treating a respiratory viral infection in a subject, the method comprising a) conducting a mass spectral analysis on a sample from the subject, wherein the presence or likelihood of a respiratory viral infection is characterized by identifying at least one spectral peak at about 38 m/z, about 51 m/z, about 55 m/z, about 59 m/z, about 69 m/z, about 77 m/z, about 94 m/z, about 103 m/z, about 109 m/z, about 122 m/z and about 143 m/z, wherein the intensity of the one or more spectral peaks, or a value derived therefrom, as compared to a reference detects the presence or predicts the likelihood of the respiratory viral infection in the subject, and b) administering a therapeutically effective amount of an anti-viral agent to the subject.

### BRIEF DESCRIPTION OF THE FIGURE

The FIGURE shows a heat map of Volatile Organic Compounds (VOCs) produced by COVID-19 positive subjects as compared to a reference

### DETAILED DESCRIPTION

The present disclosure teaches a method of detecting the presence or determining the likelihood of a respiratory viral infection in a subject.

Disclosed herein is a method of detecting the presence or determining the likelihood of a respiratory viral infection in a subject, the method comprising identifying one or more spectral peaks in a mass spectrum of a breath sample from the subject, wherein the presence or likelihood of the respiratory viral infection is characterized by identifying at least one spectral peak at about 38 m/z, about 51 m/z, about 55 m/z, about 59 m/z, about 69 m/z, about 77 m/z, about 94 m/z, about 103 m/z, about 109 m/z, about 122 m/z and about 143 m/z, wherein the intensity of the one or more spectral peaks, or a value derived therefrom, as compared to a reference detects the presence or predicts the likelihood of the respiratory viral infection in the subject.

The method as defined herein may comprise determining the likelihood of a respiratory viral infection in the subject. The term “likelihood of a respiratory viral infection” may refer to how likely it is for a respiratory viral infection to be present in a subject.

By “about” is meant a quantity, level, value, number, frequency, percentage, dimension, size, amount, weight, position or length that varies by as much as 10, 9, 8, 7, 6, 5, 4, 3, 2 or 1% to a reference quantity, level, value, number, frequency, percentage, dimension, size, amount, weight, position or length.

The terms “subject”, “patient”, “host” or “individual” used interchangeably herein, refer to any subject. The term “subject” includes any human or non-human animal. In one embodiment, the subject is a human. The term “non-human animal” includes all vertebrates, e.g., mammals and non-mammals, such as non-human primates, sheep, dog, cow, chickens, amphibians, reptiles, etc.

The term “respiratory viral infection” as used herein refers to an infection due to a respiratory virus. The term “respiratory virus” refers to a virus which infects cells of the respiratory tract, such as cells lining the oral cavity, nasopharynx, throat, larynx, bronchi and bronchioles, etc. Respiratory viruses include influenza virus, rhinovirus, adenovirus, coronavirus, respiratory syncytial virus (RSV), measles virus, mumps virus, parainfluenza virus, rubella virus, poxvirus, parvovirus, hantavirus and varicella virus.

“Coronavirus” as used herein refers to a genus in the family Coronaviridae, which family is in turn classified within the order Nidovirales. The coronaviruses are large, enveloped, positive-stranded RNA viruses. They have the largest genomes of all RNA viruses and replicate by a unique mechanism that results in a high frequency of recombination. The coronaviruses include alphacoronavirus, betacoronavirus, gammacoronavirus and deltacoronavirus. Non-limiting examples of coronaviruses include SARS coronavirus (SARS-CoV or SARS-CoV-2), MERS coronavirus, transmissible gastroenteritis virus (TGEV), human respiratory coronavirus, porcine respiratory coronavirus, canine coronavirus, feline enteric coronavirus, feline infectious peritonitis virus, rabbit coronavirus, murine hepatitis virus, sialodacryoadenitis virus, porcine hemagglutinating encephalomyelitis virus, bovine coronavirus, avian infectious bronchitis virus, and turkey coronavirus, as well as chimeras of any of the foregoing.

In one embodiment, the respiratory viral infection is a coronavirus infection. The coronavirus infection may be a COVID-19 infection (or an infection by SARS-CoV-2 virus).

In one embodiment, each spectral peak in the mass spectrum corresponds to one or more biomarkers.

The one or more biomarkers may be one or more Volatile Organic Compounds (VOCs). VOCs from the breath of a subject may be collected in a sample, e.g., on a filter, either directly or indirectly. The breath samples may be collected in a collection device which may include sorbent tubes, tedlar bags, canisters etc. It can also comprise collecting samples through a real-time breath sampler. In some embodiments, the breath sample is directly obtained from a subject at or near the laboratory or location where the biological sample will be analyzed (e.g. at an airport). In other embodiments, the breath sample may be obtained by a third party and then transferred, e.g., to a separate entity or location for analysis. In other embodiments, the sample may be obtained and tested in the same location using a point-of care test. In these embodiments, said obtaining refers to receiving the sample, e.g., from the patient, from a laboratory, from a doctor's office, from the mail, courier, or post office, etc. In some further aspects, the method may further comprise reporting the determination or test results to the subject, a health care payer, an attending clinician, a pharmacist, a pharmacy benefits manager, or any person that the determination or test results may be of interest.

The detection of the volatile organic compounds or biomarkers as defined herein may be detecting using an analytical instrument. Examples of analytical instruments include GC-MS (gas chromatography mass spectrometry), PTR-MS (proton transfer reaction mass spectrometry), SIFT-MS (selected ion flow tube mass spectrometry), sensor technologies.

In one embodiment, the presence or likelihood of a respiratory viral infection is characterized by identifying a spectral peaks selected from the group consisting of about 38 m/z, about 51 m/z, about 55 m/z, about 59 m/z, about 69 m/z, about 77 m/z, about 94 m/z, about 103 m/z, about 109 m/z, about 122 m/z and about 143 m/z.

In one embodiment, the method comprising identifying at least 1, 2, 3, 4, 5, 6, 7, 8, 9, 10, 11 or more spectral peaks in the sample.

In one embodiment, the method comprises identifying a combination of spectral peaks as shown in Table 1.

TABLE 1
List of Combinations of mass-to-charge ratios

| | Combinations of Mass-to-Charge Ratios |
|---|---|
| 1 | A method comprises identifying at least one spectral peak |
| | at about 38 m/z, about 51 m/z, about 55 m/z, about 59 m/z, |
| | about 69 m/z, about 77 m/z, about 94 m/z, about 103 m/z, |
| | about 109 m/z, about 122 m/z and about 143 m/z |
| 2 | The method according to 1, wherein the method comprises |
| | identifying at least one spectral peak at about 38 m/z. |
| 3 | The method according to 1 or 2, wherein the method |
| | comprises identifying at least one spectral peak at about 51 m/z. |
| 4 | The method according to any one of 1 to 3, wherein the |
| | method comprises identifying at least one spectral peak |
| | at about 55 m/z. |
| 5 | The method according to any one of 1 to 4, wherein the |
| | method comprises identifying at least one spectral peak |
| | at about 59 m/z. |
| 6 | The method according to any one of 1 to 5, wherein the |
| | method comprises identifying at least one spectral peak |
| | at about 69 m/z. |
| 7 | The method according to any one of 1 to 6, wherein the |
| | method comprises identifying at least one spectral peak |
| | at about 77 m/z. |
| 8 | The method according to any one of 1 to 7, wherein the |
| | method comprises identifying at least one spectral peak |
| | at about 94 m/z. |
| 9 | The method according to any one of 1 to 8, wherein the |
| | method comprises identifying at least one spectral peak |
| | at about 103 m/z. |
| 10 | The method according to any one of 1 to 9, wherein the |
| | method comprises identifying at least one spectral peak |
| | at about 109 m/z. |
| 11 | The method according to any one of 1 to 10, wherein the |
| | method comprises identifying at least one spectral peak |
| | at about 122 m/z. |
| 12 | The method according to any one of 1 to 11, wherein the |
| | method comprises identifying at least one spectral peak |
| | at about 143 m/z. |
| 13 | The method according to any one of 1 to 12, wherein the |
| | method comprises identifying at least one spectral peak. |
| 14 | The method according to any one of 1 to 12, wherein the |
| | method comprises identifying at least two spectral peaks. |
| 15 | The method according to any one of 1 to 12, wherein the |
| | method comprises identifying at least three spectral peaks. |
| 16 | The method according to any one of 1 to 12, wherein the |
| | method comprises identifying at least four spectral peaks. |
| 17 | The method according to any one of 1 to 12, wherein the |
| | method comprises identifying at least five spectral peaks. |
| 18 | The method according to any one of 1 to 12, wherein the |
| | method comprises identifying at least six spectral peaks. |
| 19 | The method according to any one of 1 to 12, wherein the |
| | method comprises identifying at least seven spectral peaks. |
| 20 | The method according to any one of 1 to 12, wherein the |
| | method comprises identifying at least eight spectral peaks. |
| 21 | The method according to any one of 1 to 12, wherein the |
| | method comprises identifying at least nine spectral peaks. |
| 22 | The method according to any one of 1 to 12, wherein the |
| | method comprises identifying at least ten spectral peaks. |
| 23 | The method according to any one of 1 to 12, wherein the |
| | method comprises identifying at least eleven spectral peaks. |

In one embodiment, the method comprises comparing the mass spectral analysis of the sample to a reference. The reference may be a subject or group of subjects without a respiratory viral infection. The reference may be a sample obtained from a subject who is healthy. The reference may also be samples obtained from a group of subjects who are healthy. Each subject may be one who does not have a respiratory viral infection, whose breath data is used as a reference.

In one embodiment, the intensity of the one or more spectral peaks, or a value derived therefrom, as compared to a reference detects the presence or predicts the likelihood of the respiratory viral infection in the subject.

As used herein, the term “increase” or “increased” with reference to a spectral peak refers to a statistically significant and measurable increase in the intensity of the spectral peak as compared to a control or reference. The increase is preferably an increase of at least about 10%, or an increase of at least about 20%, or an increase of at least about 30%, or an increase of at least about 40%, or an increase of at least about 50%.

In one embodiment, an increased level of each of the intensity of a spectral peak as compared to a reference indicates the presence of a respiratory viral infection in the subject. The increase in level may be an increase of 1.01 times, 1.02 times, 1.03 times, 1.04 times, 1.05 times, 1.06 times, 1.07 times, 1.08 times, 1.09 times, 1.1 times, 1.2 times, 1.3 times, 1.4 times, 1.5 times, 1.6 times, 1.7 times, 1.8 times, 1.9 times, 2 times, 3 times, 4 times, 5 times, 6 times, 7 times, 8 times, 9 times, 10 times, 11 times 12 times, 13 times, 14 times, 15 times, 16 times, 17 times, 18 times, 19 times, 20 times, 21 times, 22 times, 23 fold, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times,93 times, 94 times, 95 times, 96 times, 97 times, 98 times, 99 times or 100 times or anywhere in between as compared to a reference or control.

As used herein, the term “decrease” or “decreased” with reference to the intensity of a spectral peak as compared to a reference refers to a statistically significant and measurable decrease in the intensity as compared to a control or reference. The decrease is preferably a decrease of at least about 10%, or a decrease of at least about 20%, or a decrease of at least about 30%, or a decrease of at least about 40%, or a decrease of at least about 50%.

In one embodiment, a decreased level of each of the biomarker as compared to a control indicates the presence of a respiratory viral infection in the subject. The decrease in level may refer to a biomarker having 0.9 times or less, 0.8 times or less, 0.7 times or less, 0.6 times or less, 0.5 times or less, 0.4 times or less, 0.3 times or less, 0.2 times or less, 0.1 times or less or anywhere in between as compared to the level of a control.

The method as defined herein may comprise comparing the intensity of the one or more spectral peaks. The comparison of the intensity of the one or more spectral peaks can be made in comparison to one another or to a reference. The comparison of the intensity to one another may involve the determination of a ratio, wherein the value of the ratio as compared to a reference predicts the likelihood of a respiratory viral infection in the subject.

The method as defined herein may comprise determining a weighted score based on the intensity of the one or more spectral peaks and comparing it to a weighted score obtained from a control sample. Alternatively, the weighted score on the intensities of the one or more spectral peaks in the sample may be compared to a pre-determined value.

Data analysis algorithm entailing the above conditions with different weightage can be applied to determine the presence or likelihood of having a respiratory viral infection.

The method as defined herein may comprise detecting one or more biomarkers (or VOCs). It may comprise detecting a panel of biomarkers (or VOCs).

In one embodiment, the method comprising detecting at least 1, 2, 3, 4, 5, 6, 7, 8, 9, 10, 11 or more biomarkers (or VOCs) in the sample.

The step of detecting or measuring a panel of biomarkers may comprise detecting the change in the level of each biomarker in the panel of biomarkers. The change in level of a biomarker can be an increase or decrease in the level of biomarker as compared to a control or reference.

As used herein, the term “increase” or “increased” with reference to a biomarker refers to a statistically significant and measurable increase in the biomarker as compared to a control or reference. The increase is preferably an increase of at least about 10%, or an increase of at least about 20%, or an increase of at least about 30%, or an increase of at least about 40%, or an increase of at least about 50%.

In one embodiment, an increased level of each of the biomarker as compared to a control indicates the presence of a respiratory viral infection in the subject. The increase in level may be an increase of 1.01 times, 1.02 times, 1.03 times, 1.04 times, 1.05 times, 1.06 times, 1.07 times, 1.08 times, 1.09 times, 1.1 times, 1.2 times, 1.3 times, 1.4 times, 1.5 times, 1.6 times, 1.7 times, 1.8 times, 1.9 times, 2 times, 3 times, 4 times, 5 times, 6 times, 7 times, 8 times, 9 times, 10 times, 11 times 12 times, 13 times, 14 times, 15 times, 16 times, 17 times, 18 times, 19 times, 20 times, 21 times, 22 times, 23 fold, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times,93 times, 94 times, 95 times, 96 times, 97 times, 98 times, 99 times or 100 times or anywhere in between as compared to a control.

As used herein, the term “decrease” or “decreased” with reference to a biomarker refers to a statistically significant and measurable decrease in the biomarker as compared to a control or reference. The decrease is preferably a decrease of at least about 10%, or a decrease of at least about 20%, or a decrease of at least about 30%, or a decrease of at least about 40%, or a decrease of at least about 50%.

In one embodiment, a decreased level of each of the biomarker as compared to a control indicates the presence of a respiratory viral infection in the subject. The decrease in level may refer to a biomarker having 0.9 times or less, 0.8 times or less, 0.7 times or less, 0.6 times or less, 0.5 times or less, 0.4 times or less, 0.3 times or less, 0.2 times or less, 0.1 times or less or anywhere in between as compared to the level of a control.

The method as defined herein may comprise comparing the level of the one or more biomarkers. The comparison of the levels of the biomarkers can be made in comparison to one another or to a reference. The comparison of the levels to one another may involve the determination of a ratio, wherein the value of the ratio as compared to a reference predicts the likelihood of a respiratory viral infection in the subject.

The method as defined herein may comprise determining a weighted score based on the level of each biomarker in the panel of biomarkers in the sample and comparing it to a weighted score obtained from a control sample. Alternatively, the weighted score on the level of each biomarker in the panel of biomarkers in the sample may be compared to a pre-determined value.

Data analysis algorithm entailing the above conditions with different weightage can be applied to determine the presence or likelihood of having a respiratory viral infection.

As used herein, the term “sample” includes tissues, cells, body fluids and isolates thereof etc., isolated from a subject, as well as tissues, cells and fluids etc. present within a subject (i.e. the sample is in vivo). Examples of samples include: whole blood, blood fluids (e.g. serum and plasm), lymph and cystic fluids, sputum, stool, tears, mucus, hair, skin, breath (e.g. exhaled breath), ascitic fluid, cystic fluid, urine, nipple exudates, nipple aspirates, sections of tissues such as biopsy and autopsy samples, frozen sections taken for histologic purposes, archival samples, explants and primary and/or transformed cell cultures derived from patient tissues etc.

In one embodiment, the sample is a breath sample. The method as defined herein may comprise a step of obtaining a breath sample from a subject. In one embodiment, the breath sample is an exhaled breath sample. In one embodiment, the sample is end-tidal breath.

Provided herein is a sampling tube comprising breath sample as defined herein, wherein the breath sample is one that has been obtained from a subject suffering from a respiratory viral infection, and wherein the breath sample comprises a panel of biomarkers as defined herein.

In one embodiment, the method further comprises detecting one or more risk factors or symptoms in the subject. The symptom can, for example, be fever, cough, shortness of breath and/or fatigue.

In one embodiment, the method further comprises administering a therapeutically effective amount of a therapeutic agent to the subject.

The term “administering” refers to contacting, applying or providing a composition of the present invention to a subject.

The therapeutic agent for treating a coronavirus infection, such as COVID-19, include an anti-viral agent such as Remdesivir. The therapeutic agent may also comprise a therapeutic antibody such as Bamlanivimab or a combination of therapeutic antibodies comprising Casirivimab and Imdevimab.

Disclosed herein is a method of detecting the presence or determining the likelihood of a respiratory viral infection in a subject, the method comprising conducting a mass spectral analysis on a sample from the subject, wherein the presence or likelihood of a respiratory viral infection is characterized by identifying at least one spectral peak at about 38 m/z, about 51 m/z, about 55 m/z, about 59 m/z, about 69 m/z, about 77 m/z, about 94 m/z, about 103 m/z, about 109 m/z, about 122 m/z and about 143 m/z, wherein the intensity of the one or more spectral peaks, or a value derived therefrom, as compared to a reference detects the presence or predicts the likelihood of the respiratory viral infection in the subject.

The term “treating” as used herein may refer to (1) preventing or delaying the appearance of one or more symptoms of the disorder; (2) inhibiting the development of the disorder or one or more symptoms of the disorder; (3) relieving the disorder, i.e., causing regression of the disorder or at least one or more symptoms of the disorder; and/or (4) causing a decrease in the severity of one or more symptoms of the disorder.

As used herein the term “therapeutically effective amount” includes within its meaning a non-toxic but sufficient amount of an agent or compound to provide the desired therapeutic effect. The exact amount required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

Disclosed herein is a method of treating a respiratory viral infection in a subject, the method comprising a) identifying one or more spectral peaks in a mass spectrum of a breath sample from the subject, wherein the presence or likelihood of the respiratory viral infection is characterized by identifying at least one spectral peak at about 38 m/z, about 51 m/z, about 55 m/z, about 59 m/z, about 69 m/z, about 77 m/z, about 94 m/z, about 103 m/z, about 109 m/z, about 122 m/z and about 143 m/z, wherein the intensity of the one or more spectral peaks, or a value derived therefrom, as compared to a reference detects the presence or predicts the likelihood of the respiratory viral infection in the subject, and b) administering a therapeutically effective amount of an anti-viral agent to the subject.

Disclosed herein is a method of treating a respiratory viral infection in a subject, the method comprising a) conducting a mass spectral analysis on a sample from the subject, wherein the presence or likelihood of a respiratory viral infection is characterized by identifying at least one spectral peak at about 38 m/z, about 51 m/z, about 55 m/z, about 59 m/z, about 69 m/z, about 70 m/z, about 94 m/z, about 103 m/z, about 109 m/z, about 122 m/z and about 143 m/z, wherein the intensity of the one or more spectral peaks, or a value derived therefrom, as compared to a reference detects the presence or predicts the likelihood of the respiratory viral infection in the subject, and b) administering a therapeutically effective amount of an anti-viral agent to the subject.

Provided herein is a pharmaceutical composition, comprising a therapeutic agent and a pharmaceutically acceptable carrier.

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

Also disclosed herein are kits for detecting the presence or determining the likelihood of a respiratory viral infection in a subject. The kit may be configured to detect or measure a panel of biomarkers.

The articles “a” and “an” are used herein to refer to one or to more than one (i.e., to at least one) of the grammatical object of the article. By way of example, “a biomarker” means one biomarker or more than one biomarker.

As used herein, “and/or” refers to and encompasses any and all possible combinations of one or more of the associated listed items, as well as the lack of combinations when interpreted in the alternative (or).

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Certain embodiments of the invention will now be described with reference to the following examples which are intended for the purpose of illustration only and are not intended to limit the scope of the generality hereinbefore described.

### EXAMPLES

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

### Analysis Techniques

### Method 1: Gas Chromatography Mass Spectrometry (GC-MS)

### Breath Collection Procedures

The breath samples were collected using BIO-VOC sampler. The subject was asked to blow through a disposable mouth-piece till mid-tidal breath to completely displace existing environmental air in the sampling tube. The sampling tube stores a fixed volume of end-tidal breath. There is a one-way filter to prevent pathogen contamination from the air. The sampling tube is sealed with a PTFE plug. The sample was then transferred to Tenax Tube using a plunger. The tenax tube is airtight and has sorbent materials that can trap VOCs. The tenax tubes were stored at 4° C. until analysis. Each tenax tube was bar-coded and had a unique ID for identification of subjects. Three tubes of breath samples were collected form each subject and two tubes of breath were collected from each control subject. One environmental air sample was also collected to account for potential environmental contamination.

### TD-GC/MS Analysis

The analysis of VOCs was performed by Thermal Desorption-Gas Chromatography Mass Spectrometry (TD-GC/MS), using a Thermal Desorber and a GC system interfaced with a Mass Spectrometer Detector.

The sampling tube was pre-purged for 1 min and the collected breath gases were carried out at 270° C. for 10 min in the stage of primary desorption by helium gas. The total flow rate was set at 60 ml/min. The cold trap was maintained at −10° C. After primary desorption, the cold trap was rapidly heated up from −10° C. to 280° C. and hold on 5 min. The desorbed analytes were injected into a HP-5MS capillary column, via a transfer line at 120° C. The initial GC oven temperature was set at 40° C. Once the sample was injected, the oven temperature was increased to 140° C. at a rate of 5° C./min, increased to 190° C. at a rate of 20° C./min and then increased to 230° C. at a rate of 5° C./min and held for 1 min. Finally, the temperature was increased to 300° C. at a rate of 30° C./min and held for 5 mins. Helium carrier gas flow rate was 2 ml/min. Detection was achieved using MS in electron impact mode and full scan monitoring (33-550 amu). The temperature of the ion source was set at 230° C., and the quadrupole was set at 150° C. The transfer line temperature was 280° C. The gain factor was fixed at 1.58. All data files were processed by peak alignment and normalization.

### Method 2: Proton-Transfer-Reaction Mass Spectrometry (PTR-MS)

An online sampler is connected to the PTR-MS to transfer the breath sample to it in real time for analysis. This method effectively avoided sample absorption, storage and transportation, thereby minimizing sample loss and contamination. The method also enables fast on-spot detection which allows point-of-care diagnosis. The PTR-MS measures the concentration of a few hundred VOCs, some of which are disease biomarkers and will be singled out for data analysis. The peaks were identified using the library of PTR-MS, and further validated by external standards.

The instrument comprises an ionization section and a detection section. During the ionization process, the instrument forms protonated water ions (H₃O⁺) by a hollow cathode discharge in the ion source. These H₃O⁺ ions are then introduced into the drift tube by an electric drift field, where they chemically ionize the volatile organic compounds (VOC) in breath samples via proton-transfer reaction (PTR). Only the VOCs with higher proton affinity (PA) value than that of H₂O molecules will be ionized by H₃O⁺ and proceed to the detection section. These ionized VOCs are extracted by the electric field towards the time-of-flight mass spectrometer (TOF MS) to be differentiated and detected with respect to their mass-to-charge ratio (m/z). The drift tube voltage was 600V and the drift tube pressure was 2.3 mbar. The E/N ratio was 139 Td. The sampling line and buffer tube were kept at 70° C.

### EXAMPLE 1

### Methods

### Study Population

From June to August 2020, 176 patients were recruited from National Center of Infectious Disease (NCID), Tan Tock Seng Hospital Singapore (TTSH). The clinical trial in Dubai is still ongoing and we had used the clinical information of 304 patients, recruited from Nadd Al Hamar Health Center, Dubai in February and March 2021, for this report. All 480 patients were tested using the BreFence™ Go COVID-19 Breath Test System. Among the total sample size of 480 patients, 116 were COVID-19 positive while 364 were non-COVID-19 controls. Among the 116 COVID-19 positive patients, 18 were asymptomatic when they visited the clinic and the clinical information of 2 patients from the Singapore cohort were still pending. The clinical information of all the patients recruited is summarized in Table 2 below. The presence of COVID-19 infection was confirmed by the SARS-CoV-2 polymerase chain reaction (PCR) test for all patients and all control subjects tested negative in the SARS-CoV-2 PCR test. The BreFence™ Go COVID-19 Breath Test System results were compared to the Reference RT-PCR assay test results.

The clinical trial conducted in NCID was approved by National Health Group Domain Specific Review Board (NHG DSRB) to be conducted at TTSH (Ref: 2020/00579), while the clinical trial conducted in Nadd Al Hamar Health Center, Dubai was approved by the Dubai Scientific Research Ethics Committee (DSREC) of Dubai Health Authority (Ref: DSREC-01/2021_07). All enrolled patients gave signed informed consent prior to the study.

TABLE 2
Demographic suimmary of recruited subjects

| | Singapore Cohort | | Dubai Cohort | | Total | |
|---|---|---|---|---|---|---|
| | COVID | | COVID | | COVID | |
| | -19 | Control | -19 | Control | -19 | Control |
| Total Number | 17 | 159 | 99 | 205 | 116 | 364 |
| Gender (M/F) | 17/0 | 137/22 | 48/51 | 122/83 | 65/51 | 259/105 |
| Age | 32 ± 9 | 36 ± 9 | 37 ± 11 | 37 ± 13 | 37 ±11 | 37 ±12 |
| (Mean ± sdv) | | | | | | |
| Smoking | 11 | 97 | 91 | 159 | 102 | 256 |
| History | | | | | | |
| Non-smoker | | | | | | |
| Current | 4 | 29 | 8 | 44 | 12 | 73 |
| smoker | | | | | | |
| Ex-smoker | 2 | 33 | 0 | 2 | 2 | 35 |

### PTR-TOF-MS Measurement

Measurement of breath samples was done with a PTR-MS.

### Statistical Analysis of Breath VOC Data

A rough screening of the VOCs using differential analysis was performed in order to identify the metabolites that can be used to distinguish patients with and without COVID-19. A non-parametric test, Wilcoxon test, was used for differential analysis. Wilcoxon test analysis showed that 18 VOCs were significantly different between the two groups of patients who were COVID-19 positive and negative.

A decision tree model that could classify patients into the COVID-19 positive and negative groups was then built using the VOCs identified from the Wilcoxon test. In order to enhance the generalization and classification performance and obtained an optimal decision tree, the complexity of the tree and the minimum number of samples per node were limited. The complexity of the tree was 0.005 and the minimum number of samples per node was 8 samples.

Using the VOCs that were most significantly different between the positive and negative groups identified using the Wilcoxon test, 11 m/zs were found to be sufficient to build the decision tree that met the goal of obtaining at least 85% sensitivity and 95% specificity. And from the classification result, our model achieved a sensitivity of 85.34% and specificity of 96.98%.

### Results

### Clinical Performance

In this study, all 480 patients' breath samples were collected real-time and tested using BreFence™ Go COVID-19 Breath Test System together with a nasopharyngeal (NP) swab for reverse transcription polymerase chain reaction (RT-PCR) testing. The BreFence™ Go test results were compared to the Reference RT-PCR assay test results.

### Identity of VOCs

The 11 mass-to-charge ratio (m/z) that were used in the decision tree model were identified and summarized in Table 3.

TABLE 3
Identity of selected VOCs associated with COVID-19

| S/N | mass-to-charge ratio (m/z) |
|---|---|
| 1 | 38.04 |
| 2 | 51.04 |
| 3 | 55.05 |
| 4 | 59.05 |
| 5 | 69.08 |
| 6 | 77.06 |
| 7 | 94.07 |
| 8 | 103.08 |
| 9 | 109.07 |
| 10 | 121.96 |
| 11 | 143.18 |

The following clauses were included in the priority application SG 10202109466S and are included as part of the description of this application.

Clause 1. A method of detecting the presence or determining the likelihood of a respiratory viral infection in a subject, the method comprising identifying one or more spectral peaks in a mass spectrum of a breath sample from the subject, wherein the presence or likelihood of the respiratory viral infection is characterized by identifying at least one spectral peak at about 38 m/z, about 51 m/z, about 55 m/z, about 59 m/z, about 69 m/z, about 77 m/z, about 94 m/z, about 103 m/z, about 109 m/z, about 122 m/z and about 143 m/z, wherein the intensity of the one or more spectral peaks, or a value derived therefrom, as compared to a reference detects the presence or predicts the likelihood of the respiratory viral infection in the subject.

Clause 2. The method of 1, wherein a likelihood of a respiratory viral infection is characterized by identifying a spectral peaks selected from the group consisting of about 38 m/z, about 51 m/z, about 55 m/z, about 59 m/z, about 69 m/z, about 77 m/z, about 94 m/z, about 103 m/z, about 109 m/z, about 122 m/z and about 143 m/z.

Clause 3. The method of 1 or 2, wherein the method comprising detecting at least 1, 2, 3, 4, 5, 6, 7, 8, 9, 10, 11 or more spectral peaks in the sample.

Clause 4. The method of any one of 1 to 3, wherein the method comprises comparing the mass spectral analysis of the sample to a reference. Clause 5. The method of any one of 1 to 4, wherein the sample is a breath sample.

Clause 6. The method of 5, wherein the breath sample is an exhaled breath sample.

Clause 7. The method of any one of 1 to 6, wherein the method further comprises detecting one or more risk factors or symptoms in the subject.

Clause 8. The method of any one of 1 to 7, wherein the respiratory viral infection is a coronavirus infection.

Clause 9. The method of 8, wherein the coronavirus infection is a COVID-19 infection.

Clause 10. The method of any one of 1 to 9, wherein the method further comprises administering a therapeutically effective amount of a therapeutic agent to the subject.

Clause 11. A method of detecting the presence or determining the likelihood of a respiratory viral infection in a subject, the method comprising conducting a mass spectral analysis on a sample from the subject, wherein the presence or likelihood of a respiratory viral infection is characterized by identifying at least one spectral peak at about 38 m/z, about 51 m/z, about 55 m/z, about 59 m/z, about 69 m/z, about 77 m/z, about 94 m/z, about 103 m/z, about 109 m/z, about 122 m/z and about 143 m/z, wherein the intensity of the one or more spectral peaks, or a value derived therefrom, as compared to a reference detects the presence or predicts the likelihood of the respiratory viral infection in the subject.

Clause 12. A method of treating a respiratory viral infection in a subject, the method comprising a) identifying one or more spectral peaks in a mass spectrum of a breath sample from the subject, wherein the presence or likelihood of the respiratory viral infection is characterized by identifying at least one spectral peak at about 38 m/z, about 51 m/z, about 55 m/z, about 59 m/z, about 69 m/z, about 77 m/z, about 94 m/z, about 103 m/z, about 109 m/z, about 122 m/z and about 143 m/z, wherein the intensity of the one or more spectral peaks, or a value derived therefrom, as compared to a reference detects the presence or predicts the likelihood of the respiratory viral infection in the subject, and b) administering a therapeutically effective amount of an anti-viral agent to the subject.

Clause 13. A method of treating a respiratory viral infection in a subject, the method comprising a) conducting a mass spectral analysis on a sample from the subject, wherein the presence or likelihood of a respiratory viral infection is characterized by identifying at least one spectral peak at about 38 m/z, about 51 m/z, about 55 m/z, about 59 m/z, about 69 m/z, about 77 m/z, about 94 m/z, about 103 m/z, about 109 m/z, about 122 m/z and about 143 m/z, wherein the intensity of the one or more spectral peaks, or a value derived therefrom, as compared to a reference detects the presence or predicts the likelihood of the respiratory viral infection in the subject, and b) administering a therapeutically effective amount of an anti-viral agent to the subject.

What is claimed is:

1. A method of treating a respiratory viral infection in a subject, the method comprising:
(i) identifying one or more spectral peaks in a mass spectrum of a breath sample from the subject, wherein the presence of the respiratory viral infection is characterized by identifying at least one spectral peak at about 38 m/z, about 51 m/z, about 55 m/z, about 59 m/z, about 69 m/z, about 77 m/z, about 94 m/z, about 103 m/z, about 109 m/z, about 122 m/z and about 143 m/z, wherein the intensity of the one or more spectral peaks, or a value derived therefrom, as compared to a reference, detects the presence of the respiratory viral infection in the subject; and
(ii) administering a therapeutically effective amount of an anti-viral agent to the subject.

2. The method of claim 1, wherein the presence of the respiratory viral infection is characterized by identifying spectral peaks selected from the group consisting of about 38 m/z, about 51 m/z, about 55 m/z, about 59 m/z, about 69 m/z, about 77 m/z, about 94 m/z, about 103 m/z, about 109 m/z, about 122 m/z and about 143 m/z.

3. The method of claim 1, wherein step (i) of the method comprising detecting at least 1, 2, 3, 4, 5, 6, 7, 8, 9, 10, 11 or more spectral peaks in the sample.

4. The method of claim 1, wherein step (i) of the method comprises comparing the mass spectral analysis of the sample to a reference.

5. The method of claim 1, wherein the sample is a breath sample.

6. The method of claim 5, wherein the breath sample is an exhaled breath sample.

7. The method of claim 1, wherein step (i) of the method further comprises detecting one or more risk factors or symptoms in the subject.

8. The method of claim 1, wherein the respiratory viral infection is a coronavirus infection.

9. The method of claim 8, wherein the coronavirus infection is a COVID-19 infection.

10. A method of treating a respiratory viral infection in a subject, the method comprising:
(i) conducting a mass spectral analysis on a sample from the subject, wherein the presence of a respiratory viral infection is characterized by identifying at least one spectral peak at about 38 m/z, about 51 m/z, about 55 m/z, about 59 m/z, about 69 m/z, about 77 m/z, about 94 m/z, about 103 m/z, about 109 m/z, about 122 m/z and about 143 m/z, wherein the intensity of the one or more spectral peaks, or a value derived therefrom, as compared to a reference detects the presence of the respiratory viral infection in the subject; and
(ii) administering a therapeutically effective amount of an anti-viral agent to the subject.
